(19) United States
(12) Patent Application Publication
(10) Pub. No.: US 2014/0017677 A1
(43) Pub. Date: Jan. 16, 2014

(54) METHOD FOR ESTABLISHMENT OF PERSONALITY-GENOTYPE CORRELATION MODEL AND APPLICATION THEREOF

(71) Applicant: GENOMICS BIOSCI & TECH. CO., LTD, New Taipei City (TW)

(72) Inventors: Chao-Yuan Yu, New Taipei City (TW); Pei-Ling Wang, New Taipei City (TW); Su-Lien Chen, New Taipei City (TW)

(73) Assignee: GENOMICS BIOSCI & TECH. CO., LTD, New Taipei City (TW)

(21) Appl. No.: 13/799,487

(22) Filed: Mar. 13, 2013

(30) Foreign Application Priority Data
Jul. 12, 2012 (TW) 101125171

(51) Int. Cl. C12Q 1/68

(52) U.S. Cl. CPC C12Q 1/6876
USPC 435/6.11

(57) ABSTRACT

The present invention discloses a method using gene detection to analyze personality traits of a human subject, which comprising steps of (a) providing a subject's test specimen; (b) conducting a gene polymorphism analysis for a plurality of genetic polymorphism biomarkers of the test specimen, wherein the plurality of genetic polymorphism biomarkers are composed of following single-nucleotide polymorphisms (SNPs); TPH1(SEQ ID NO:2), EGF(SEQ ID NO:4), NET_T-182C(SEQ ID NO:5), DRD1(SEQ ID NO:8), DRD4(SEQ ID NO:10) and COMT(SEQ ID NO:11), and following variable number tandem repeats (VNTRs): MAOA_VNTR(SEQ ID NO:16); and (c) determining the subject's surgerncy, negative affection and orienting/regulation of personality traits according to the results of the gene polymorphism analysis; wherein TPH1, DRD4 and COMT are correlated to surgency, EGF and DRD1, MAOA_VNTR are correlated to negative affection, and NET_T-182C and COMT are correlated to orienting/regulation of personality traits.

### CROSS-REFERENCES TO RELATED APPLICATIONS

This application claims the priority of Taiwanese patent application No. 101125171, filed on Jul. 12, 2012, which is incorporated herewith by reference.

### BACKGROUND OF THE INVENTION

1. Field of the Invention

The present invention relates to a method for establishment of personality traits detection model and its applications, especially relates to a method using genetic detection technique to establish a personality-genotype correlation model and application thereof.

2. The Prior Arts

The inherited personality traits of an individual have been a topic that attracts many researchers. Each kind of human personality trait has its own uniqueness. Because of the interactions among these various personality traits, the society is plentiful, diverse, tough yet also gentle, and harmonized. Personality traits consist of an individual's inclination and personality psychology. The personality traits are expressed features of an individual's intrinsic and stable psychological characteristics that including mainly three dimensions, namely personality, temperament and ability, and among these features, the personality is the core feature of a person's personality psychology. Personality not only interacts with temperament and ability, but also these three features constrain each other. In addition, personality has the feature of emotion and determination. Personality theory commonly proposes that personality of an individual is different from one another and that personality can be measured. Today commonly used personality questionnaires include Myers-Briggs Type Indicator (MBTI), Enneagram Personality Test, Multiple Factor Personality Test, DISC Personality Test, Minnesota Multiple Personality Inventory (MMPI), California Psychology Inventory (CPI), and 16 Personality Factor Questionnaire (16 PF), etc.

Currently commonly used personality measurement tools are the character test and personality test. However, analysis results obtained from these tests are not centralized, inconvenient, and non-searchable, therefore, many problems have arisen when current personality evaluation tools are used. These shortcomings include lack of accuracy and efficiency, abused design of evaluation questionnaire, misuse of evaluation questionnaire, lack of professionals in the implementation of personality evaluation test, and exaggeration of results of personality profile.

Development of human neurological function reaches the level of mature adults at age five to seven. This stage is also known to be the best period for enhancement of children's physiological and mental development. Furthermore, behavior genetics studies have shown that temperament and personality traits are significantly impacted by genes inherited. It is estimated that about 40 to 70 percentage of characters such as activity level, anxiety, emotion, social activity, job preference, and control model are impacted by gene inherited. Thomas and Chess (1977, 1986 and 1989) has proposed a cross interaction model that emphasizes the complimentary action of cognition and education. Therefore, inherited personality traits of an infant can be enhanced, induced, or interfered by parents, family and macro-environmental factors.

Preschool to school age is widely recognized as the golden stage of growth and development in human life. Irrespective of desirable or negative characters, these personality traits are indispensable and can not be justified as good or bad in its essence. Therefore, it is important to accept and understand the nature of a child, and provide he/she appropriate treatment and education based on individual needs at different development stage. If suitable stimulation and training are provided, it is possible to stimulate potentials of a child effectively.

### DETAILED DESCRIPTION OF THE PREFERRED EMBODIMENT

Because the traditional methods used for analysis of personality traits are insufficient, development of a genetic analysis tool to analyze an individual's personality traits becomes a new trend.

Therefore, a purpose of the present invention is to provide a genetic method for analysis of an individual's personality traits, comprising the steps of (a) providing a subject's test specimen; (b) conducting a gene polymorphism analysis for a plurality of genetic polymorphism biomarkers of the test specimen, wherein the plurality of genetic polymorphism biomarkers are composed of following single-nucleotide polymorphisms (SNPs); TPH1(SEQ ID NO:2), EGF(SEQ ID NO:4), NET_T-182C(SEQ ID NO:5), DRD1(SEQ ID NO:8), DRD4(SEQ ID NO:10) and COMT(SEQ ID NO:11), and following variable number tandem repeats (VNTRs): MAOA_VNTR(SEQ ID NO:16); and (c) determining the subject's surgerncy, negative affection and orienting/regulation of personality traits according to the results of the gene polymorphism analysis; wherein TPH1 (SEQ ID NO:2), DRD4 (SEQ ID NO:10) and COMT (SEQ ID NO:11) are correlated to surgency, EGF (SEQ ID NO:4) and DRD1 (SEQ ID NO:8), MAOA_VNTR(SEQ ID NO:16) are correlated to negative affection, and NET_T-182C (SEQ ID NO:5) and COMT (SEQ ID NO:11) are correlated to orienting/regulation of personality traits.

Preferably, the genotype AA of TPH1 (SEQ ID NO:2) is correlated to surgency, and genotype CC of TPH1 (SEQ ID NO:2) is correlated to surgency, perceptual sensitivity, high intensity pleasure, and vocal reativity of personality traits; the genotype GG of DRD (SEQ ID NO:10) is correlated to perceptual sensitivity of personality traits; the genotype CC of COMT (SEQ ID NO:11) is correlated to perceptual sensitivity and the genotype GG of COMT (SEQ ID NO:11) is correlated to cuddliness of personality traits, the genotype AG of DRD1 (SEQ ID NO:8) is correlated to negative affection of personality trait, the genotype CC of EGF (SEQ ID NO: 5) is correlated to duration of orienting of personality trait, the genotype AG of EGF (SEQ ID NO:4) is correlated to falling reactivity/rate of recover, the genotype 3R3R of MAOA_VNTR (SEQ ID NO: 16) is correlated to sadness of personality trait. and the subject is an infant.

Another purpose of the present invention is to provide a method for establishment of a model of personality and genotype relationship, including the steps of (a) implementing a questionnaire survey for a subject; (b) providing the subject's specimen to conduct a gene polymorphism analysis that is specific for a plurality of genetic polymorphism biomarkers, and (c) implementing a statistic analysis for the results of the questionnaire survey and the gene polymorphism analysis, and accordingly establishing a personality-gene correlation model.

Preferably, the plurality of genetic polymorphism biomarkers are selected from the following groups consisting of CYP2C19*2 (SEQ ID NO:1), TPH1 (SEQ ID NO:2), BDNF (SEQ ID NO:3), EGF (SEQ ID NO:4), NET_T-182C (SEQ ID NO:5), NET_A-3081T (SEQ ID NO:6), EAAT2 (SEQ ID NO:7), DRD1 (SEQ ID NO:8), DRD2 (SEQ ID NO:9), DRD4 (SEQ ID NO:10), COMT (SEQ ID NO:11), and CYP17A1 (SEQ ID NO:12), and the following variable number tandem repeats (VNTRs) consisting of 5HTT_VNTR (SEQ ID NO:13), 5HTTstin2_VNTR (SEQ ID NO:14), DRD4_VNTR (SEQ ID NO:15), MAOA_VNTR (SEQ ID NO:16). The personality traits comprise surgency, negative affection and orienting/regulation, and the subject is an infant.

The approach of the present invention is to use the genetic detection method to analyze personality traits of a child and the genetic analysis results can be a foundation of personality analysis of a child. The genetic detection method of the present invention is convenient as compared to traditional personality traits determination methods, and the genetic analysis method can be broadly applied to the new born or a fetus even at the stage of early pregnancy. Furthermore, the systematic research method based on the genotype and questionnaire statistic results can be used to establish highly reliable personality genotype model of a child. This genotype model not only can compensate the deficiency and disadvantages observed in the traditional questionnaire method, but can be a professional foundation of potential commercial product. Derivative products and services of this personality genotype detection method can be children's personality genetic analysis kit and adaptive education program designed for a specific genotype personality. By integration of the pre-school children personality genetic determination and adaptive education program resource, parents and teachers can correctly assess a child's personality genotype, and know more about impact of family, environment, and education on a child growth and reduce cost and useless efforts of trial error. Moreover, this method can be combined with expert advice and appropriate education planning to provide parents good advices for children's future education and career planning, such that good parent-children relationship may be established, educational environment may be improved. Ultimately, a child can benefit from this method to have a good development and self-realization.

### EXAMPLES

The concept of the present invention is mainly focused on the application of genetic detection on analysis of infant personality traits, and use of the genetic detection as a core foundation of infant personality analysis. Although genetic testing to mature, however most of its application is focused on disease detection, but less in living applications. Furthermore, personality traits analysis method remains mainly relying on questionnaire and interview. When a subject is a child, professionals have to spend more time on observation to make right justification. Therefore, it is conceivable that genetic detection method will be more convenient than the traditional determination method, and the genetic method can be applied even at the early pregnancy stage. Moreover, the method of the present invention, based on genotype correlation research and systematic statistical analysis, can be used for establishment of highly reliable model of child personality trait analysis, which not only may compensate deficiency of traditional questionnaire but can serve as a professional foundation of future commercially valuable products.

Accordingly, the present invention provides a method for establishment of a personality-genotype correlation model, including the steps of (a) implementing a questionnaire survey for a subject; (b) providing the subject's specimen to conduct a gene polymorphism analysis that is specific for a plurality of genetic polymorphism biomarkers, and (c) implementing a statistic analysis for the results of the questionnaire survey and the gene polymorphism analysis, and accordingly establishing a personality-genotype correlation model. The specific implementation processes of these steps are detailed as follows.

The above mentioned method is based on literature review, questionnaire survey, and genetic analysis technique to develop genotype testing technology designed for children's personality analysis and consequently to construct the correlation model of genotypes associated with personality traits. Using evidence-based and systematic research approaches, inadequacy and deficiencies of traditional personality analysis method can be amended. In addition, combination of highly accurate genetic detection method can improve reliability of personality analysis results. Thus, the technical specifications of the present invention are as follows.

1. Selection of Candidate Genes Associated with Children's Personality Traits

Genes that potentially associated with children's personality traits are identified through literature review and database search. As for the secondary data collection and analysis, first the secondary data are collected and studies. Then technology related to gene testing of personality traits of Taiwan and international children, intellectual properties such as patent applied and granted, and business models are reviewed. Finally, candidate genes linked to personality polymorphism and SNPs are proposed.

### 2. Gene Testing Technique of Children Personality Traits

In order to identify method suitable for children's personality traits identification, experts of children education are recruited to conduct questionnaire survey, sample collection, and study design. The clinical trial protocol is submitted to Institution Review Board (IRB) for review. After IRB approval, 200 children specimens are collected and questionnaire surveys are conducted. Soft brushes are used to collect children's test specimen and these samples are shipped back to laboratory for analysis following standard operation procedures, including filing out every subject's information and establishing barcode file, high quality nucleotide extraction and purification, candidate gene PCR reaction, agarose gel electrophoresis, nucleotide sequencing and decoding, sequencing code analysis and comparison, etc., to develop professional and accurate child genotype detection technique.

### 3. Establishment of Correlation Model of Children Personality Traits and Genotype

The present invention uses two infant temperament scales, one called “Infant Temperament Questionnaire” (abbreviated as ITQ) and another called “The Infant Behavior Questionnaire” (abbreviated as IBQ). The results of infant temperament scales and the results of children personality gene detection are entered in professional biomedical statistics analysis software (such as SAS, SPSS) and the data are evaluated by professional bio-statisticians in depth in order to establish correlation between children personality traits and genotype. The present model includes a series of gene polymorphism locus, distribution frequency of these loci and decision model. The analytical results are specific and unique and contribute to establishment of personality trait analysis standard.

### Infant Temperament Questionnaire

### Editor

This questionnaire used in the present invention is prepareded by Children's Mental Health Center of National Taiwan University Hospital, a modification and amendment questionnaire edited by Carey and McDevitt (1980), the Infant Temperament Questionnaire (abbreviated as ITQ). The questionnaire is applicable to assess the infants of 4-8 months.

### Content of Questionnaire

This weigh table of the questionnaire is divided into two parts, in which the first part is for the basic information of participant infants and the second part is the scale content. The scale content has nine dimensions, including activity level, regularity, approach, adaptability, attention span, persistence, emotion nature, reactivity threshold, and reaction level and a total of 95 questions.

### Scoring Method

The scoring method adopts five-point Likert Scale, score is assigned from 1 to 5 (never to very frequent) by assessment of frequency of the subject's behavior. If a (−) marking is shown, it suggests a negative statement and negative score is given. High score indicates higher corresponding of a specific personality trait of the tested subject infant. For example, higher score in activity level item indicates that the infant has higher activity level and vise versa.

### Reliability

In terms of reliability, internal consistency reliability is in the range of 0.49 to 0.71. In terms of validity, the results should provide constructive feedback.

### Infant Behavior Questionnaire

### Editor

Rothbart and Garstein revised the infant behavior questionnaire of 3 to 12 month-old infant in 2003. The score is revised to become 14 scale table with 191 questions. In Taiwan, Ms. Lei has translated the questionnaire into Chinese.

### Content

The content of the questionnaire includes activity level, distress to limitations, approach, fear, duration of orienting, smiling and laughter, vocal reactivity, sadness, perceptual sensitivity, high intensity pleasure, low intensity pleasure, cuddliness, soothability, and falling reactivity/rate of recover.

Based on 2003 revision of Rothbart and Gartstein's questionnaire, IBQ-R factor analysis evaluates three factors, including (1) surgency/extraversion, that is, approach, vocal reactivity, high intensity pleasure, smiling and laughter, activity level, and perceptual sensitivity; (2) negative affectivity, that is, sadness, distress to limitations, fear, loading negatively, and falling reactivity/rate of recover; and (3) orienting/regulation, that is, low intensity pleasure, cuddliness, duration of orienting, and soothability. Coefficient of Cronbach's alpha of these three factors is 0.92, 0.91 and 0.91 respectively.

Reliability and validity: Reliability coefficient is between 0.77 to 0.89, with constructive validity.

### Reliability

Adjust the experimental conditions in accordance with the selected candidate genes and build genotype identification platform. The collected specimens are then subjected to genetic polymorphism identification. In addition, the collected questionnaires are divided into the experimental group and the control group and then case-control association study is conducted in order to establish correlation model of personality traits and genotypes. All gene detection results and statistical modeling results shall undergo infant personality gene detection integration test, and it is required that overall analysis process meets relevant standards.

### Results

### Enrollment of Clinical Trial Subjects and Questionnaire Scale Analysis

A total of 205 subjects were enrolled, of which 12 participants dropped out this plan. Therefore, to total collected effective questionnaire were 193.

IBQ scale results is evaluated by dividing into three aspects and fourteen sub-items. Cronbach's alpha reliability coefficients of these three aspects are 0.92, 0.91, and 0.91 respectively, and the results provide constructive feedback.

Surgency: approach, vocal reactivity, high intensity pleasure, smiling and laughter, activity level, perceptual sensitivity

Negative Affection: sadness, distress to limitations, fear, falling reactivity/rate of recover.

Orienting/regulation: low intensity pleasure, cuddliness, duration of orienting, soothability.

### Selection of Gene Marker Candidate

Through literature research, sixteen gene markers that are potentially associated with personality traits are identified, as shown in Table 1.

TABLE 1

| Type of Gene Marker | Name of Gene Marker | SEQ ID NO |
|---|---|---|
| SNP | CYP2C19*2 | SEQ ID NO: 1 |
| | TPH1 | SEQ ID NO: 2 |
| | BDNF | SEQ ID NO: 3 |
| | EGF | SEQ ID NO: 4 |
| | NET(T-182C) | SEQ ID NO: 5 |
| | NET(A-3081T) | SEQ ID NO: 6 |
| | EAAT2 | SEQ ID NO: 7 |
| | DRD1 | SEQ ID NO: 8 |
| | DRD2 | SEQ ID NO: 9 |
| | DRD4 | SEQ ID NO: 10 |
| | COMT | SEQ ID NO: 11 |
| | CYP17A1 | SEQ ID NO: 12 |
| VNTR | 5HTT_VNTR | SEQ ID NO: 13 |
| | 5HTTstin2_VNTR | SEQ ID NO: 14 |
| | DRD4_VNTR | SEQ ID NO: 15 |
| | MAOA_VNTR | SEQ ID NO: 16 |

### Establishment of Child Genotype Identification Platform

### Barcode System for Clinical Enrollment

Every enrolled subject is assigned with a unique barcode using a computer software tool. The barcode is printed out and used to label the questionnaire and sampling brush. Barcode reader is also set up to facilitate access of scale results of questionnaire evaluation and genetic data.

### Quality Control Process of Nucleotide Extraction and Purification

Oral mucosa saliva samples of four to eight-month-old infants (in total 200 infants) are collected using sterile soft brush. The collected samples are subjected to the following nucleotide gDNA extraction and purification processes.

The sampling brush containing collected specimen is immersed in 0.6 ml cell lysis buffer for 2 hours. During the treatment, the brush holder are scrubbed again the test tube to allow the cells completely released from the brush and treated by the cell lysis buffer. Proteinase-K (20 mg/ml) is then added, mixed and followed by dry heat treatment at 56° C. for 2 hours. 0.5 ml Tris-HC1 buffer (pH 8.0) is then added to dissolve DNA.

Because nucleotides absorb at 260 nm, therefore, the purity of gDNA is assessed by absorbance of the nucleotide sample at 260 and 280 nm, the ratio of absorbance at 260 nm and 280 nm and the nucleotide concentration using spectrophotometer.

### PCR Product Detection and Quality Control

### Design and Synthesis of High Specificity Primers

Target gene sequences are selected from the GeneBank and Primer3plus software is used to design primers. In general, it is preferred that Tm value of the designed primers is in the range of 56˜62° C., GC content not exceeding 50%, and primer length in the range of 18 to 22mers. However, the most suitable, highly specific primers can only be determined and selected when they have undergone tests following the PCR quality control procedures described below. Those non-specific primers are eliminated during studies. Primer synthesis are outsourced and conducted by the well-known company, IDT (Integrated DNA Technologies, Inc). Quality of each synthesized primer is controlled by quantitative analysis and by stringent MALDI-TOF Mass Spectrometry analysis. Those primers not meeting designated quality requirement shall be re-synthesized until they pass all tests as required. Primer DNA sequence of each target gene marker and the size of PCR products are shown in Table 2.

### Polymerase Chain Reaction (PCR) and Agarose Gel Electrophoresis Analysis

Every PCR solution contains 1.5 mM MgCl₂, 10 mM Tris-HCl (pH8.3), 50 mM KCl, 0.4 mM dNTPs, 1 unit of Taq polymerase, 0.50M Primer pair, and 10 ng of template DNA. Run a gradient temperature program to determine optimal temperature for primer annealing (Ta). Running the PCR reaction following the program: 95° C. for 5 mins (95° C. for 30 seconds; Ta° C. for 30 seconds; 72° C. for 30 seconds; Ta temperature varies with each individual gene) for 40 cycles, and 72° C. for 7 mins When the PCR reaction is completed, PCR products are subjected to 2% agarose gel electrophoresis analysis to determine if the amplification process is successful, signal strength is normal, and if the product is specific enough, etc. The process is fine tuned to obtain optimal reaction condition. The extracted and purified 200 DNA specimens are amplified following the PCR reaction process described above.

### Nucleotide Sequencing Reaction and Sequencing Analysis

ExoASP enzyme mixture is used to eliminate primer and dNTPs left in the PRC product. The treated PCR product is then subjected to DNA sequencing. Each sequencing reaction solution contains 1× Buffer, 0.5 uL of BigDye v3.1, 0.5 uM of primer specifically for sequencing, and 10 ng of ExoSAP-ed PCR product. Running the DNA sequencing following the program: 96° C. for 10 seconds; 55° C. for 5 seconds, 60° C. for 4 minutes, and repeated 40 cycles until sequencing program end. EDTA and ethanol are added to eliminate excess fluorescent agents in the reaction solution and the reaction product is purified. Finally, the sequencing product is re-dissolved in HiDi-formamide and then treated at 96° C. for 3 minutes to allow the product becoming single strand structure. ABI3730 DAN analyzer is used to decode gene sequence and AB DNA Sequencing Analysis Software v5.2 is used to perform DNA sequence quality analysis. The acceptance criteria are QV≧20, S/N ratio≧50, and fluorescence signal ≧300. If the product quality does meet quality requirement, such as high background value, many noise signals, weak signal, high fluorescence agent residual, or presence of secondary structure, etc., sequencing program must be modified to meet the designated sequencing quality criteria (QV≧20, S/N ratio≧50, and fluorescence signal ≧300).

### Fluorescent Fragment Analysis

PCR products of MAOA VNTR, 5HTT-48 bpVNTR, 5HTT-17 bp VNTR, DRD4 VNTR fluorescent fragments are diluted and mixed with HiDi-formamide and size standard (LIZ600). These solutions are subjected to ABI3730 DNA analyzer for capillary electrophoresis analysis. Set up Bin Set parameters such that Data are analyzed using AB GeneMapper Software v4.0 for genotype analysis.

### Genotype Justification

Enter SNP Database of NCBI to search gene variants information, for example rs number, flanking sequence, and allele frequency etc. Then CodonCode Aligner software is used to analyze SNP mutations. Detailed genotype justification of each individual gene marker is shown in the appendix.

TABLE 2

| | | Primer-F | Primer-R | PCR |
|---|---|---|---|---|
| | Genbank ID/ | (Forward primer)/ | (reverse primer)/ | product |
| Biomearker | SEQ ID NO | SEQ ID NO | SEQ ID NO | (bp) |
| CYP2C19*2 | rs4244285/ | CCAGAGCTTGGCATATTGTA/ | GTCCATCGATTCTTGGTGT/ | 198 |
| | SEQ ID NO: 1 | SEQ ID NO: 17 | SEQ ID NO: 18 | |
| TPH1 | rs1800532/ | TTTTTGGTGTGCGAGGATTA/ | TTTCCCCCACTGGAATACAA/ | 221 |
| | SEQ ID NO: 2 | SEQ ID NO: 19 | SEQ ID NO: 20 | |
| BDNF | rs6265/ | CAAACATCCGAGGACAAGGT/ | CCTCATGGACATGTTTGCAG/ | 305 |
| | SEQ ID NO: 3 | SEQ ID NO: 21 | SEQ ID NO: 22 | |
| EGF | rs4444903/ | AAAGGAGGTGGAGCCTGAAG/ | AGGGAAGCCACAGGAAAGAT/ | 311 |
| | SEQ ID NO: 4 | SEQ ID NO: 23 | SEQ ID NO: 24 | |
| NET(T-182C) | rs2242446/ | GTGAGTTCAATCCCAGCCAT/ | AGGAACTTTACCGGTCCTGG/ | 326 |
| | SEQ ID NO: 5 | SEQ ID NO: 25 | SEQ ID NO: 26 | |
| NET(A-3081T) | rs28386840/ | CCACACCCCTTATCATCCAC/ | TGTTGGGTTTGGTGTCTTTG/ | 463 |
| | SEQ ID NO: 6 | SEQ ID NO: 27 | SEQ ID NO: 28 | |
| EAAT2 | rs4354668/ | GTGATGTCAGCTCTCGACGAA/ | CTGCCACCTGTGCTTTGCT/ | 326 |
| | SEQ ID NO: 7 | SEQ ID NO: 29 | SEQ ID NO: 30 | |
| DRD1 | rs4532/ | AGCAAGGGAGTCAGAAGACAGA/ | GTGTTCAGAGTCCTCATCTTCCT/ | 254 |
| | SEQ ID NO: 8 | SEQ ID NO: 31 | SEQ ID NO: 32 | |
| DRD2 | rs1800497/ | ACGGCTCCTTGCCCTCTAG/ | CCTTCCTGAGTGTCATCAAC/ | 236 |
| | SEQ ID NO: 9 | SEQ ID NO: 33 | SEQ ID NO: 34 | |
| DRD4 | rs1800955/ | AGGATCAACTGTGCAACGG/ | TGGTATCTGGCAAAACCTCC/ | 250 |
| | SEQ ID NO: 10 | SEQ ID NO: 35 | SEQ ID NO: 36 | |
| COMT | rs4680/ | ATCCAAGTTCCCCTCTCTC/ | CTTTTTCCAGGTCTGACAAC/ | 290 |
| | SEQ ID NO: 11 | SEQ ID NO: 37 | SEQ ID NO: 38 | |
| CYP17A1 | rs743572/ | GCTCCAGGAGAATCTTTC/ | GACAATCACTGTAGTCTTGG/ | 378 |
| | SEQ ID NO: 12 | SEQ ID NO: 39 | SEQ ID NO: 40 | |
| 5HTT_VNTR | 48 bp VNTR, | FAM-GGCGTTGCCGCTCTG | GAGGGACTGAGCTGGACA | 528 or |
| | Promoter/SEQ | AATGC/ | ACCAC/ | 484 |
| | ID NO: 13 | SEQ ID NO: 41 | SEQ ID NO: 42 | |
| 5HTTstin2_VNTR | 17 bp VNTR, | FAM-GCTGTGGACCTGGGCAATGT/ | GACTGAGACTGAAAAGACATAATC/ | 12R = |
| | Intron 2/ | SEQ ID NO: 43 | SEQ ID NO: 44 | 334 |
| | SEQ ID NO: 14 | | | |
| DRD4_VNTR | 48 bp VNTR, | FAM -CTGCAGCGCTGGGA | AGGACCCTCATGGCCTTG/ | 4R = |
| | Exon 3/ | GGTG | SEQ ID NO: 46 | 389 |
| | SEQ ID NO: 15 | SEQ ID NO: 45 | | |
| MAOA_VNTR | 30 bpVNTR | TET-AGCACGCGTGCCTCA | CCGAGATTCGGCGGGCCCT | 140, |
| | (MAOA-uVNTR)/ | GCCTCCTTCCCCGGC/ | CCGCCTTGCGC | 170, |
| | SEQ ID NO: 16 | SEQ ID NO: 47 | SEQ ID NO: 48 | 200, |
| | | | | 230 |

Results of test sample SNP and VNTR genotype determination is shown in the attachment, the table below only shows results of frequency of each genotype (as shown in tables 3-18).

TABLE 3
5HTT_VNTR

| | | | | | Cumulative |
|---|---|---|---|---|---|
| | | Frequency | Percent | Valid Percent | Percent |
| Valid | SS | 100 | 48.8 | 51.5 | 51.5 |
| | SL | 77 | 37.6 | 39.7 | 91.2 |
| | LL | 17 | 8.3 | 8.8 | 100.0 |
| | Total | 194 | 94.6 | 100.0 | |

| | | |
|---|---|---|
| Missing 999 | 11 | 5.4 |
| Total | 205 | 100.0 |

TABLE 4
MAOA_VNTR

| | | | | | Cumulative |
|---|---|---|---|---|---|
| | | Frequency | Percent | Valid Percent | Percent |
| Valid | 2R2R | 2 | 1.0 | 1.0 | 1.0 |
| | 2R3R | 3 | 1.5 | 1.5 | 2.5 |
| | 3R3R | 84 | 41.0 | 41.8 | 44.3 |
| | 3R4R | 62 | 30.2 | 30.8 | 75.1 |
| | 4R4R | 50 | 24.4 | 24.9 | 100.0 |
| | Total | 201 | 98.0 | 100.0 | |

| | | |
|---|---|---|
| Missing 999 | 4 | 2.0 |
| Total | 205 | 100.0 |

TABLE 5
5HTTstin2_VNTR

| | | | | | Cumulative |
|---|---|---|---|---|---|
| | | Frequency | Percent | Valid Percent | Percent |
| Valid | 8R12R | 1 | .5 | .5 | .5 |
| | 10R12R | 90 | 43.9 | 44.6 | 45.0 |
| | 12R12R | 111 | 54.1 | 55.0 | 100.0 |
| | Total | 202 | 98.5 | 100.0 | |

| | | |
|---|---|---|
| Missing 999 | 3 | 1.5 |
| Total | 205 | 100.0 |

TABLE 6
DRD4_VNTR

| | | | | | Cumulative |
|---|---|---|---|---|---|
| | | Frequency | Percent | Valid Percent | Percent |
| Valid | 2R2R | 8 | 3.9 | 4.2 | 4.2 |
| | 2R3R | 1 | .5 | .5 | 4.7 |
| | 2R4R | 63 | 30.7 | 33.0 | 37.7 |
| | 2R5R | 1 | .5 | .5 | 38.2 |
| | 2R7R | 1 | .5 | .5 | 38.7 |
| | 4R4R | 105 | 51.2 | 55.0 | 93.7 |
| | 4R5R | 6 | 2.9 | 3.1 | 96.9 |
| | 4R6R | 5 | 2.4 | 2.6 | 99.5 |
| | 4R7R | 1 | .5 | .5 | 100.0 |
| | Total | 191 | 93.2 | 100.0 | |

| | | |
|---|---|---|
| Missing 999 | 14 | 6.8 |
| Total | 205 | 100.0 |

TABLE 7
TPH1

| | | | | | Cumulative |
|---|---|---|---|---|---|
| | | Frequency | Percent | Valid Percent | Percent |
| Valid | AA | 46 | 22.4 | 22.5 | 22.5 |
| | AC | 90 | 43.9 | 44.1 | 66.7 |
| | CC | 68 | 33.2 | 33.3 | 100.0 |
| | Total | 204 | 99.5 | 100.0 | |

| | | |
|---|---|---|
| Missing 999 | 1 | .5 |
| Total | 205 | 100.0 |

TABLE 8
EGF

| | | | | | Cumulative |
|---|---|---|---|---|---|
| | | Frequency | Percent | Valid Percent | Percent |
| Valid | AA | 23 | 11.2 | 11.3 | 11.3 |
| | AG | 76 | 37.1 | 37.3 | 48.5 |
| | GG | 105 | 51.2 | 51.5 | 100.0 |
| | Total | 204 | 99.5 | 100.0 | |

| | | |
|---|---|---|
| Missing 999 | 1 | .5 |
| Total | 205 | 100.0 |

TABLE 9
NET_T182C

| | | | | | Cumulative |
|---|---|---|---|---|---|
| | | Frequency | Percent | Valid Percent | Percent |
| Valid | CC | 21 | 10.2 | 10.3 | 10.3 |
| | CT | 92 | 44.9 | 45.3 | 55.7 |
| | TT | 90 | 43.9 | 44.3 | 100.0 |
| | Total | 203 | 99.0 | 100.0 | |

| Missing 999 | 2 | 1.0 |
|---|---|---|
| Total | 205 | 100.0 |

TABLE 10
NET_A3081T

| | | | | | Cumulative |
|---|---|---|---|---|---|
| | | Frequency | Percent | Valid Percent | Percent |
| Valid | AA | 59 | 28.8 | 29.8 | 29.8 |
| | AT | 97 | 47.3 | 49.0 | 78.8 |
| | TT | 42 | 20.5 | 21.2 | 100.0 |
| | Total | 198 | 96.6 | 100.0 | |

| | | |
|---|---|---|
| Missing 999 | 7 | 3.4 |
| Total | 205 | 100.0 |

TABLE 11
DRD2

| | | | | | Cumulative |
|---|---|---|---|---|---|
| | | Frequency | Percent | Valid Percent | Percent |
| Valid | CC | 75 | 36.6 | 36.9 | 36.9 |
| | CT | 93 | 45.4 | 45.8 | 82.8 |
| | TT | 35 | 17.1 | 17.2 | 100.0 |
| | Total | 203 | 99.0 | 100.0 | |

| | | |
|---|---|---|
| Missing 999 | 2 | 1.0 |
| Total | 205 | 100.0 |

TABLE 12
COMT

| | | | | | Cumulative |
|---|---|---|---|---|---|
| | | Frequency | Percent | Valid Percent | Percent |
| Valid | AA | 20 | 9.8 | 9.8 | 9.8 |
| | AG | 78 | 38.0 | 38.2 | 48.0 |
| | GG | 106 | 51.7 | 52.0 | 100.0 |
| | Total | 204 | 99.5 | 100.0 | |

| | | |
|---|---|---|
| Missing 999 | 1 | .5 |
| Total | 205 | 100.0 |

TABLE 13
DRD4_A521G

| | | | | | Cumulative |
|---|---|---|---|---|---|
| | | Frequency | Percent | Valid Percent | Percent |
| Valid | AA | 89 | 43.4 | 43.4 | 43.4 |
| | AG | 97 | 47.3 | 47.3 | 90.7 |
| | AG | 97 | 47.3 | 47.3 | 90.7 |
| | GG | 19 | 9.3 | 9.3 | 100.0 |
| | Total | 205 | 100.0 | 100.0 | |

TABLE 14
CYP17A1

| | | | | | Cumulative |
|---|---|---|---|---|---|
| | | Frequency | Percent | Valid Percent | Percent |
| Valid | CC | 65 | 31.7 | 31.9 | 31.9 |
| | CT | 104 | 50.7 | 51.0 | 82.8 |
| | TT | 35 | 17.1 | 17.2 | 100.0 |
| | Total | 204 | 99.5 | 100.0 | |

| | | |
|---|---|---|
| Missing 999 | 1 | .5 |
| Total | 205 | 100.0 |

TABLE 15
BDNF

| | | | | | Cumulative |
|---|---|---|---|---|---|
| | | Frequency | Percent | Valid Percent | Percent |
| Valid | CC | 44 | 21.5 | 21.6 | 21.6 |
| | CT | 96 | 46.8 | 47.1 | 68.6 |
| | TT | 64 | 31.2 | 31.4 | 100.0 |
| | Total | 204 | 99.5 | 100.0 | |

| | | |
|---|---|---|
| Missing 999 | 1 | .5 |
| Total | 205 | 100.0 |

TABLE 16
CYP2C19_2

| | | | | | Cumulative |
|---|---|---|---|---|---|
| | | Frequency | Percent | Valid Percent | Percent |
| Valid | AA | 20 | 9.8 | 9.8 | 9.8 |
| | AG | 101 | 49.3 | 49.5 | 59.3 |
| | GG | 83 | 40.5 | 40.7 | 100.0 |
| | Total | 204 | 99.5 | 100.0 | |

| | | |
|---|---|---|
| Missing 999 | 1 | .5 |
| Total | 205 | 100.0 |

TABLE 17
DRD1

| | | | | | Cumulative |
|---|---|---|---|---|---|
| | | Frequency | Percent | Valid Percent | Percent |
| Valid | AA | 140 | 68.3 | 69.7 | 69.7 |
| | AG | 54 | 26.3 | 26.9 | 96.5 |
| | GG | 7 | 3.4 | 3.5 | 100.0 |
| | Total | 201 | 98.0 | 100.0 | |

| | | |
|---|---|---|
| Missing 999 | 4 | 2.0 |
| Total | 205 | 100.0 |

TABLE 18
EAAT2

| | | | | | Cumulative |
|---|---|---|---|---|---|
| | | Frequency | Percent | Valid Percent | Percent |
| Valid | | 41 | 16.7 | 17.4 | 17.4 |
| | AA | 21 | 8.5 | 8.9 | 26.3 |
| | AC | 62 | 25.2 | 26.3 | 52.5 |
| | CC | 112 | 45.5 | 47.5 | 100.0 |
| | Total | 236 | 95.9 | 100.0 | |

| | | |
|---|---|---|
| Missing 999 | 10 | 4.1 |
| Total | 246 | 100.0 |

### Results of Relationship Questionnaire Evaluation and Genotype Analysis

The relationship of questionannaire scale and the genotype is analyzed using Bonferroni correction of SPSS software. The results listed in below table shows statistic <0.5.

DRD1 Genotype AG and Negative Affectivity are correlated (as shown in tables 19-20).

TABLE 19
Correlation of DRD1 vs Three Major Personality Traits

| | DRD1 | | | | | | | |
|---|---|---|---|---|---|---|---|---|
| | | | AA | | AG | | GG | |
| | | Standard | | Standard | | Standard | | Standard |
| | Mean | Deviation | Mean | Deviation | Mean | Deviation | Mean | Deviation |
| Surgency | — | — | 4.15 | 1.02 | 4.08 | 1.02 | 3.21 | .93 |
| Negative | — | — | 3.65 | .51 | 3.76 | .46 | 3.27 | .26 |
| affectivity | | | | | | | | |
| Orienting | — | — | 4.40 | .75 | 4.51 | .73 | 4.33 | 1.03 |

TABLE 20
Comparisons of Column Means^{a}

DRD1

| | | | |
|---|---|---|---|
| | AA | AG | GG |
| (A) | (B) | (C) | (D) |

| | | |
|---|---|---|
| Surgency | . | |
| Negative affectivity | . | D |
| Orienting | . | |

TPH1 Genotype AA and CC are correlated to Surgency (as shown in tables 21-22).

TABLE 21
Correlation of TPH1 vs Three Major Personality Traits

| | TPH1 | | | | | | | |
|---|---|---|---|---|---|---|---|---|
| | | | AA | | AC | | CC | |
| | | Standard | | Standard | | Standard | | Standard |
| | Mean | Deviation | Mean | Deviation | Mean | Deviation | Mean | Deviation |
| Surgency | — | — | 4.35 | 1.04 | 3.79 | 1.02 | 4.33 | .93 |
| Negative | — | — | 3.76 | .50 | 3.57 | .46 | 3.74 | .51 |
| affectivity | | | | | | | | |
| Orienting | — | — | 4.60 | .85 | 4.32 | .74 | 4.45 | .68 |

TABLE 22
Comparisons of Column Means^{a}

TPH1

| | | | |
|---|---|---|---|
| | AA | AC | CC |
| (A) | (B) | (C) | (D) |

| | | | |
|---|---|---|---|
| Surgency | . | C | C |
| Negative affectivity | . | | |
| Orienting | . | | |

DRD4 (A-521G) Genotype GG is correlated to Perceptual Sensitivity (as shown in tables 23-24).

TABLE 23
Correlation of DRD4(A-521G) Genotype and Perceptual Sensitivity

| | DRD4_A521G | | | | | | | |
|---|---|---|---|---|---|---|---|---|
| | | | AA | | AG | | GG | |
| | | Standard | | Standard | | Standard | | Standard |
| | Mean | Deviation | Mean | Deviation | Mean | Deviation | Mean | Deviation |
| Activity | — | — | 3.66 | .90 | 3.78 | .87 | 3.93 | .82 |
| Level | | | | | | | | |
| Approach | — | — | 3.20 | 1.16 | 3.10 | 1.12 | 3.56 | 1.21 |
| Smiling and | — | — | 3.56 | 3.73 | 1.32 | 1.65 | 3.81 | 1.14 |
| Laughter | | | | | | | | |
| Vocal | — | — | 3.22 | 1.12 | 3.27 | 1.05 | 3.90 | 1.22 |
| Reactivity | | | | | | | | |
| High | — | — | 4.05 | 1.51 | 3.92 | 1.42 | 4.64 | 1.32 |
| Intensity | | | | | | | | |
| Pleasure) | | | | | | | | |
| Perceptual | — | — | 2.48 | 1.05 | 2.50 | 1.11 | 3.22 | .83 |
| Sensitivity | | | | | | | | |

TABLE 24
Comparisons of Column Means^{a}

DRD4_A521G

| | | | |
|---|---|---|---|
| | AA | AG | GG |
| (A) | (B) | (C) | (D) |

| | | |
|---|---|---|
| Activity Level | . | |
| Approach | . | |
| Smiling and Laughter | . | |
| Vocal Reactivity | . | |
| High Intensity Pleasure | . | |
| Perceptual Sensitivity | . | B C |

COMT Genotype AA is correlated to Perceptual Sensitivity (as shown in tables 25-26).

TABLE 25
Correlation between COMT and Perceptual Sensitivity

| | COMT | | | | | | | |
|---|---|---|---|---|---|---|---|---|
| | | | AA | | AG | | GG | |
| | | Standard | | Standard | | Standard | | Standard |
| | Mean | Deviation | Mean | Deviation | Mean | Deviation | Mean | Deviation |
| Activity | — | — | 3.50 | .74 | 3.86 | .93 | 3.71 | .86 |
| Level | | | | | | | | |
| Approach | — | — | 3.25 | .81 | 3.22 | 1.24 | 3.16 | 1.14 |
| Smiling and | — | — | 3.60 | 1.24 | 3.57 | 1.28 | 3.75 | 1.64 |
| Laughter | | | | | | | | |
| Vocal | — | — | 3.11 | 1.04 | 3.32 | 1.26 | 3.34 | 1.01 |
| Reactivity | | | | | | | | |
| High | — | — | 4.25 | 1.30 | 3.91 | 1.53 | 4.11 | 1.44 |
| Intensity | | | | | | | | |
| Pleasure) | | | | | | | | |
| Perceptual | — | — | 3.17 | 1.03 | 2.50 | 1.09 | 2.49 | 1.05 |
| Sensitivity | | | | | | | | |

TABLE 26
Comparisons of Column Means^{a}

COMT

| | | | |
|---|---|---|---|
| | AA | AG | GG |
| (A) | (B) | (C) | (D) |

| | | |
|---|---|---|
| Activity Level | . | |
| Approach | . | |
| Smiling and Laughter | . | |
| Vocal Reactivity | . | |
| High Intensity Pleasure | . | |
| Perceptual Sensitivity | . | D |

TPH1 Genotype CC is correlated to Perceptual Sensitivity, High Intensity Pleasure, Vocal Reactivity (as shown in tables 27-28).

TABLE 27
Correlation Between TPH1 and Perceptual Sensitivity,
High Intensity Pleasure, and Vocal Reactivity

| | TPH1 | | | | | | | |
|---|---|---|---|---|---|---|---|---|
| | | | AA | | AC | | CC | |
| | | Standard | | Standard | | Standard | | Standard |
| | Mean | Deviation | Mean | Deviation | Mean | Deviation | Mean | Deviation |
| Activity | — | — | 3.81 | .93 | 3.61 | .82 | 3.87 | .89 |
| Level | | | | | | | | |
| Approach | — | — | 3.41 | 1.23 | 2.98 | 1.18 | 3.31 | 1.01 |
| Smiling and | — | — | 4.09 | 2.09 | 3.37 | 1.23 | 3.75 | 1.16 |
| Laughter | | | | | | | | |
| Vocal | — | — | 3.49 | 1.05 | 3.02 | 1.09 | 3.55 | 1.10 |
| Reactivity | | | | | | | | |
| High | — | — | 4.34 | 1.48 | 3.70 | 1.42 | 4.28 | 1.44 |
| Intensity | | | | | | | | |
| Pleasure | | | | | | | | |
| Perceptual | — | — | 2.62 | 1.06 | 2.27 | 1.05 | 2.88 | 1.03 |
| Sensitivity | | | | | | | | |

TABLE 28
Comparisons of Column Means^{a}

TPH1

| | | | |
|---|---|---|---|
| | AA | AC | CC |
| (A) | (B) | (C) | (D) |

| | | |
|---|---|---|
| Activity Level | . | |
| Approach | . | |
| Smiling and Laughter | . | |
| Vocal Reactivity | . | C |
| High Intensity Pleasure | . | C |
| Perceptual Sensitivity | . | C |

MAOA Genotype 3R/3R is correlated Negative Affectivity—-Sadness (as shown in tables 29-30).

TABLE 29

| | MAOA_VNYR | | | | | | | | | |
|---|---|---|---|---|---|---|---|---|---|---|
| | 2R2R | | 2R3R | | 3R3R | | 3R4R | | 4R4R | |
| | | Standard | | Standard | | Standard | | Standard | | Standard |
| | Mean | Deviation | Mean | Deviation | Mean | Deviation | Mean | Deviation | Mean | Deviation |
| Sadness | 4.47 | .56 | 3.81 | 1.16 | 3.33 | .91 | 2.88 | .86 | 3.27 | .91 |
| Fear | 3.41 | .40 | 3.27 | .54 | 3.46 | .83 | 3.27 | .65 | 3.59 | .91 |
| Falling | 4 | 1.74 | 5.13 | .69 | 4.53 | 1.03 | 4.76 | .76 | 4.45 | 1.01 |
| reactivity | | | | | | | | | | |

TABLE 30
Comparisons of Column Meansa

MAOA_VNYR

| | | | | |
|---|---|---|---|---|
| 2R2R | 2R3R | 3R3R | 3R4R | 4R4R |
| (A) | (B) | (C) | (D) | (E) |

| | |
|---|---|
| Sadness | E |
| Fear | |
| Falling reactivity | |

EFG Genotype A/G is correlated to Negative Affectivity—-Falling Reactivity (as shown in tables 31-32).

TABLE 31
Correlation Between EGF and Negative Affectivity

| | EGF | | | | | | | |
|---|---|---|---|---|---|---|---|---|
| | | | AA | | AG | | GG | |
| | | Standard | | Standard | | Standard | | Standard |
| | Mean | Deviation | Mean | Deviation | Mean | Deviation | Mean | Deviation |
| Sadness | — | — | 3.21 | .86 | 3.31 | .94 | 3.16 | .92 |
| Fear | — | — | 3.62 | .86 | 3.46 | .76 | 3.38 | .82 |
| Falling | — | — | 4.11 | .83 | 4.70 | .99 | 4.60 | .93 |
| reacivity | | | | | | | | |

TABLE 32
Comparisons of Column Means^{a}

EGF

| | | | |
|---|---|---|---|
| | AA | AG | GG |
| (A) | (B) | (C) | (D) |

| | | |
|---|---|---|
| Sadness | . | |
| Fear | . | |
| Falling reactivity | . | B |

CYP2C19*2 Genotype A/A is correlated to Negative Affectivity—-Sadness (as shown in tables 33-34).

TABLE 33
Correlation Between CYP2C19*2 and Negative Affectivity

| | CYP2C19_2 | | | | | | | |
|---|---|---|---|---|---|---|---|---|
| | | | AA | | AG | | GG | |
| | | Standard | | Standard | | Standard | | Standard |
| | Mean | Deviation | Mean | Deviation | Mean | Deviation | Mean | Deviation |
| Sadness | — | — | 3.70 | .94 | 3.24 | .95 | 3.09 | .83 |
| Fear | — | — | 3.80 | .90 | 3.47 | .80 | 3.33 | .76 |
| Falling | — | — | 4.46 | 1.12 | 4.48 | .93 | 4.72 | .95 |
| reacivity | | | | | | | | |

TABLE 34
Comparisons of Column Means^{a}

CYP2C19_2

| | | | |
|---|---|---|---|
| | AA | AG | GG |
| (A) | (B) | (C) | (D) |

| | | |
|---|---|---|
| Sadness | . | D |
| Fear | . | |
| Falling reactivity | . | |

NET(T-182C) Genotype C/C is correlated to Regulation Behavior -Duration of Orienting (as shown in tables 35-36).

TABLE 35
Correlation Between NET(T-182C) and Orienting Regulation Behavior

| | NET_T182C | | | | | | | |
|---|---|---|---|---|---|---|---|---|
| | | | CC | | CT | | TT | |
| | | Standard | | Standard | | Standard | | Standard |
| | Mean | Deviation | Mean | Deviation | Mean | Deviation | Mean | Deviation |
| Low | — | — | 4.32 | 1.13 | 3.90 | 1.24 | 4.21 | 1.35 |
| Intensity | | | | | | | | |
| Pleasure | | | | | | | | |
| Cuddliness | — | — | 5.91 | .52 | 5.85 | .75 | 5.83 | .74 |
| Duration | — | — | 3.52 | 1.39 | 2.68 | 1.27 | 3.16 | 1.35 |
| of | | | | | | | | |
| Orienting | | | | | | | | |
| Soothability | — | — | 5.08 | .87 | 4.82 | .86 | 4.83 | .84 |

TABLE 36
Comparisons of Column Means^{a}

NET_T182C

| | | | |
|---|---|---|---|
| | CC | CT | TT |
| (A) | (B) | (C) | (D) |

| | | |
|---|---|---|
| Low Intensity Pleasure | . | |
| Cuddliness | . | |
| Duration of Orienting | . | C |
| Soothability | . | |

COMT Genotype G/G is correlated to Regulation Behavior—Cuddliness (as shown in tables 37-38).

TABLE 37
Correlation Between COMT Orienting Regulation Behavior

| | COMT | | | | | | | |
|---|---|---|---|---|---|---|---|---|
| | | | AA | | AG | | GG | |
| | | Standard | | Standard | | Standard | | Standard |
| | Mean | Deviation | Mean | Deviation | Mean | Deviation | Mean | Deviation |
| Low | — | — | 4.02 | 1.24 | 4.08 | 1.36 | 4.08 | 1.25 |
| Intensity | | | | | | | | |
| Pleasure | | | | | | | | |
| Cuddliness | — | — | 5.47 | .78 | 5.86 | .75 | 5.91 | .68 |
| Duration | — | — | 3.53 | 1.15 | 2.90 | 1.26 | 2.92 | 1.41 |
| of | | | | | | | | |
| Orienting | | | | | | | | |
| Soothability | — | — | 4.67 | .68 | 4.93 | .90 | 4.79 | .84 |

TABLE 38
Comparisons of Column Means^{a}

COMT

| | | | |
|---|---|---|---|
| | AA | AG | GG |
| (A) | (B) | (C) | (D) |

| | | |
|---|---|---|
| Low Intensity Pleasure | . | |
| Cuddliness | . | B |
| Duration of Orienting | . | |
| Soothability | . | |

Based on the results of infant gene and questionnaire scale screening technology platform, in total eight gene markers are identified in correlation to personality traits, and children personality traits can be grouped into three major aspects.

### I) Surgency TPH1(Genotye AA and CC)

i) perceptual sensitivity DRD4 (Genotype GG),
COMT (Genotype AA)
TPH1 (Genotype CC)

ii) high intensity pleasure TPH1 (Genotype CC)

iii) vocal reactivity TPH1 (Genotype CC)

### II) Negative Affection DRD 1 (Genotype AG)

i) sadness MAOA_VNTR (Genotype 3R3R)

ii) falling reactivity EGF (Genotype AG)

### III) Orienting

i) duration of orienting NET (T-182C) (Genotype CC)

ii) cuddliness COMT (Genotype GG)

According to examples of the present invention, sample number can be accumulated up to 1000 persons in the future, thus reliability of the personality traits genotype grouping database can be enhanced. In the meantime number of candidate gene markers will increase such that discrimination level of personality trait genotype grouping database can be elevated.

Furthermore, another example is provided according to the above results, in which a gene detection method used for analysis of personality traits comprises of the steps of (a) providing a subject's test specimen; (b) conducting a gene polymorphism analysis for a plurality of genetic polymorphism biomarkers of the test specimen, wherein the plurality of genetic polymorphism biomarkers are composed of following single-nucleotide polymorphisms (SNPs); TPH1(SEQ ID NO:2), EGF(SEQ ID NO:4), NET_T-182C(SEQ ID NO:5), DRD1(SEQ ID NO:8), DRD4(SEQ ID NO:10) and COMT(SEQ ID NO:11), and following variable number tandem repeats (VNTRs): MAOA_VNTR(SEQ ID NO:16); and (c) determining the subject's surgerncy, negative affection and orienting/regulation of personality traits according to the results of the gene polymorphism analysis; wherein TPH1 (SEQ ID NO:2), DRD4 (SEQ ID NO:10) and COMT (SEQ ID NO:11) are correlated to surgency, EGF (SEQ ID NO:4) and DRD1 (SEQ ID NO:8), MAOA_VNTR(SEQ ID NO:16) are correlated to negative affection, and NET_T-182C (SEQ ID NO:5) and COMT (SEQ ID NO:11) are correlated to orienting/regulation of personality traits.

Through the present invention derivative products and services of this personality genotype detection method can be children's personality genetic analysis kit and adaptive education program designed for a specific genotype personality. By integration of the pre-school children personality genetic determination and adaptive education program resource, parents and teachers can correctly assess a child's personality genotype, and know more about impact of family, environment, and education on a child growth and reduce cost and useless efforts of trial error. Moreover, this method can be combined with expert advice and appropriate education planning to provide parents good advices for children's future education and career planning, such that good parent-children relationship may be established, educational environment may be improved. Ultimately, a child can benefit from this method to have a good development and self-realization.

From the examples abovementioned the present invention, a gene detection method for analysis of personality traits, provides value of industrial application. The present invention is further explained in the following embodiment illustration and examples. Those examples below should not, however, be considered to limit the scope of the invention, it is contemplated that modifications will readily occur to those skilled in the art, which modifications will be within the spirit of the invention and the scope of the appended claims.

What is claimed is:

1. A gene detection method for analysis of personality, comprising the steps of:
(a) providing a subject's test specimen;
(b) conducting a gene polymorphism analysis for a plurality of genetic polymorphism biomarkers of the test specimen, wherein the plurality of genetic polymorphism biomarkers are composed of following single-nucleotide polymorphisms (SNPs); TPH1(SEQ ID NO:2), EGF(SEQ ID NO:4), NET_T-182C(SEQ ID NO:5), DRD1(SEQ ID NO:8), DRD4(SEQ ID NO:10) and COMT(SEQ ID NO:11), and following variable number tandem repeats (VNTRs): MAOA_VNTR(SEQ ID NO:16); and
(c) determining the subject's surgerncy, negative affection and orienting /regulation of personality traits according to the results of the gene polymorphism analysis;
wherein TPH1 (SEQ ID NO:2), DRD4 (SEQ ID NO:10) and COMT (SEQ ID NO:11) are correlated to surgency, EGF (SEQ ID NO:4) and DRD1 (SEQ ID NO:8), MAOA_VNTR(SEQ ID NO:16) are correlated to negative affection, and NET_T-182C (SEQ ID NO:5) and COMT (SEQ ID NO:11) are correlated to orienting/regulation of personality traits.

2. The method as claimed in claim 1, wherein the genotype AA of TPH1 (SEQ ID NO:2) is correlated to surgency, and genotype CC of TPH1 (SEQ ID NO:2) is correlated to surgency, perceptual sensitivity, high intensity pleasure, and vocal reativity of personality traits.

3. The method as claimed in claim 1, wherein the genotype GG of DRD (SEQ ID NO:10) is correlated to perceptual sensitivity of personality traits.

4. The method as claimed in claim 1, wherein the genotype CC of COMT (SEQ ID NO:11) is correlated to perceptual sensitivity and the genotype GG of COMT (SEQ ID NO:11) is correlated to cuddliness of personality traits.

5. The method as claimed in claim 1, wherein the genotype AG of DRD1 (SEQ ID NO:8) is correlated to negative affection of personality trait.

6. The method as claimed in claim 1, wherein the genotype CC of EGF (SEQ ID NO: 5) is correlated to duration of orienting of personality trait.

7. The method as claimed in claim 1, wherein the genotype AG of EGF (SEQ ID NO:4) is correlated to falling reactivity/rate of recover.

8. The method as claimed in claim 1, wherein the genotype 3R3R of MAOA_VNTR (SEQ ID NO: 16) correlated to sadness of personality trait.

9. The method as claimed in claim 1, wherein the subject is an infant.

10. A method for establishment of personality-genotype correlation model, comprising steps of:
(a) implementing a questionnaire survey for a subject;
(b) providing the subject's specimen to conduct a gene polymorphism analysis that is specific for a plurality of genetic polymorphism biomarkers, and
(c) implementing a statistic analysis for the results of the questionnaire survey and the gene polymorphism analysis, and accordingly establishing a personality-genotype correlation model.

11. The method as claimed in claim 10, wherein the plurality of genetic polymorphism biomarkers are selected from the following groups consisting of CYP2C19*2 (SEQ ID NO:1), TPH1 (SEQ ID NO:2), BDNF (SEQ ID NO:3), EGF (SEQ ID NO:4), NET_T-182C (SEQ ID NO:5), NET_A-3081T (SEQ ID NO:6), EAAT2 (SEQ ID NO:7), DRD1 (SEQ ID NO:8), DRD2 (SEQ ID NO:9), DRD4 (SEQ ID NO:10), COMT (SEQ ID NO:11), and CYP17A1 (SEQ ID NO:12), and the following variable number tandem repeats (VNTRs) consisting of 5HTT_VNTR (SEQ ID NO:13), 5HTTstin2_VNTR (SEQ ID NO:14), DRD4_VNTR (SEQ ID NO:15), MAOA_VNTR (SEQ ID NO:16).

12. The method as claimed in claim 10, wherein the personality traits comprise surgency, negative affection and orienting/regulation, surgency includes perceptual sensitivity, high intensity pleasure, and vocal reactivity, negative affection includes sadness and falling reactivity/rate of recover, and the orienting/regulation includes duration of orienting and cuddliness.

13. The method as claimed in claim 10, wherein the human subject is an infant.


SEQUENCE LISTING (48 sequences)

SEQ ID NO: 1 - DNA, 52 bp, Homo sapiens
  allele (1)..(52): r=a or g
tttcccactatcattgattatttcccrggaacccataacaaattacttaaaa

SEQ ID NO: 2 - DNA, 52 bp, Homo sapiens
  allele (1)..(52): m=a or c
attattaattgacaacctattaggtgmtagctgctattctgagcatagggaa

SEQ ID NO: 3 - DNA, 52 bp, Homo sapiens
  allele (1)..(52): SNP rs6265; r=a or g
catcattggctgacactttcgaacacrtgatagaagagctgttggatgagga

SEQ ID NO: 4 - DNA, 52 bp, Homo sapiens
  allele (1)..(52): SNP rs4444903; r=a or g
tctttcagccccaatccaagggttgtrgctggaactttccatcagttcttcc

SEQ ID NO: 5 - DNA, 52 bp, Homo sapiens
  allele (1)..(52): SNP rs2242446; y=c or t
tccaaatttttccagcggacgcgcgcycttttctgggaaccctgcgtccgct

SEQ ID NO: 6 - DNA, 52 bp, Homo sapiens
  allele (1)..(52): SNP rs28386840; w=a or t
cgaggcccccaccagggctgagcaccwgtttccccagcagcaagtgctcctt

SEQ ID NO: 7 - DNA, 52 bp, Homo sapiens
  allele (1)..(52): SNP rs4354668; k=g or t
tgcgcgtgtgcgggtgtgtgcgcgcckggggaggcggtggaggccgctgcgc

SEQ ID NO: 8 - DNA, 52 bp, Homo sapiens
  allele (1)..(52): SNP rs4532; y=c or t
aggggctctgacacccctcaagttccyaagcagggaataggggtcagtcaga

SEQ ID NO: 9 - DNA, 52 bp, Homo sapiens
  allele (1)..(52): SNP rs1800497; y=c or t
ctggacgtccagctgggcgcctgcctygaccagcactttgaggatggctgtg

SEQ ID NO: 10 - DNA, 52 bp, Homo sapiens
  allele (1)..(52): SNP rs1800955; y=c or t
ggggcagggggagcgggcgtggagggygcgcacgaggtcgaggcgagtccgc

SEQ ID NO: 11 - DNA, 52 bp, Homo sapiens
  allele (1)..(52): SNP rs4680; r=a or g
cccagcggatggtggatttcgctggcrtgaaggacaaggtgtgcatgcctga

SEQ ID NO: 12 - DNA, 52 bp, Homo sapiens
  allele (1)..(52): SNP rs743572; r=a or g
gggtgccggcaggcaagatagacagcrgtggagtagaagagctgtggcaact

SEQ ID NO: 13 - DNA, 529 bp, Homo sapiens
  satellite (1)..(529): 48bp VNTR
ggcgttgccgctctgaatgccagcacctaacccctaatgtccctactgcagccctcccagcatcccccctgcaacctcccagcaactccctgtacccctcctaggatcgctcctgcatcccccattatcccccccttcacccctcgcggcatcccccctgcacccccagcatcccccctgcagcccccccagcatctcccctgcacccccagcatcccccctgcagcccttccagcatccccctgcacctctcccaggatctcccctgcaacccccattatcccccctgcacccctcgcagtatcccccctgcaccccccagcatccccccatgcacccccggcatcccccctgcacccctccagcattctccttgcaccctaccagtattcccccgcatcccggcctccaagcctcccgcccaccttgcggtccccgccctggcgtctaggtggcaccagaatcccgcgcggactccacccgctgggagctgccctcgcttgcccgtggttgtccagctcagtccctc

SEQ ID NO: 14 - DNA, 334 bp, Homo sapiens
  satellite (1)..(334): 17bp VNTR
gctgtggacctgggcaatgtctggcgcttcccctacatatgttaccagaatggagggggtcagtatcacaggctgcgagtagaggctgtgacccagggtgggctgtgacccggagtgggctgtgacccggggtgggctgtgacccgggtgggctgcgacctggggtgggctgtgacctgggatgggctgtgacccgggtgggctgtgacctggggtgggctgtgacccgggtgggctgtgacctggggtgggctgtgacccgggtgggctgtgacctgggatgggctgtaggtcctcttgagaggccagaagacagattatgtcttttcagtct

SEQ ID NO: 15 - DNA, 389 bp, Homo sapiens
  satellite (1)..(389): 48bp VNTR
ctgcagcgctgggaggtggcacgtcgcgccaagctgcacggccgcgcgccccgccgacccagcggccctggcccgccttcccccacgccacccgcgccccgcctcccccaggacccctgcggccccgactgtgcgccccccgcgcccggccttccccggggtccctgcggccccgactgtgcgcccgccgcgcccagcctcccccaggacccctgcggccccgactgtgcgccccccgcgcccggcctccccccggacccctgcggctccaactgtgctccccccgacgccgtcagagccgccgcgctcccaccccagactccaccgcagacccgcaggaggcggcgtgccaagatcaccggccgggagcgcaaggccatgagggtcct

SEQ ID NO: 16 - DNA, 170 bp, Homo sapiens
  satellite (1)..(170): 30bpVNTR
agcacgcgtgcctcagcctccttccccggcggcaccggcaccggcaccagtacccgcaccagtaccggcaccggcaccagtacccgcaccagtaccggcaccggcaccagtacccgcaccagtaccggcaccggcaccgagcgcaaggcggagggcccgccgcgcaaggc

SEQ ID NO: 17 - DNA, 20 bp, artificial sequence
  misc_feature (1)..(20): forward primer of SNP rs4244285
ccagagcttggcatattgta

SEQ ID NO: 18 - DNA, 19 bp, artificial sequence
  misc_feature (1)..(19): reverse primer of SNP rs4244285
gtccatcgattcttggtgt

SEQ ID NO: 19 - DNA, 20 bp, artificial sequence
  misc_feature (1)..(20): forward primer of SNP rs1800532
tttttggtgtgcgaggatta

SEQ ID NO: 20 - DNA, 20 bp, artificial sequence
  misc_feature (1)..(20): reverse primer of SNP rs1800532
tttcccccactggaatacaa

SEQ ID NO: 21 - DNA, 20 bp, artificial sequence
  misc_feature (1)..(20): forward primer of SNP rs6265
caaacatccgaggacaaggt

SEQ ID NO: 22 - DNA, 20 bp, artificial sequence
  misc_feature (1)..(20): reverse primer of SNP rs6265
cctcatggacatgtttgcag

SEQ ID NO: 23 - DNA, 20 bp, artificial sequence
  misc_feature (1)..(20): forward primer of SNP rs4444903
aaaggaggtggagcctgaag

SEQ ID NO: 24 - DNA, 20 bp, artificial sequence
  misc_feature (1)..(20): reverse primer of SNP rs4444903
agggaagccacaggaaagat

SEQ ID NO: 25 - DNA, 20 bp, artificial sequence
  misc_feature (1)..(20): forward primer of SNP rs2242446
gtgagttcaatcccagccat

SEQ ID NO: 26 - DNA, 20 bp, artificial sequence
  misc_feature (1)..(20): reverse primer of SNP rs2242446
aggaactttaccggtcctgg

SEQ ID NO: 27 - DNA, 20 bp, artificial sequence
  misc_feature (1)..(20): forward primer of SNP rs28386840
ccacaccccttatcatccac

SEQ ID NO: 28 - DNA, 20 bp, artificial sequence
  misc_feature (1)..(20): reverse primer of SNP rs28386840
tgttgggtttggtgtctttg

SEQ ID NO: 29 - DNA, 21 bp, artificial sequence
  misc_feature (1)..(21): forward primer of SNP rs4354668
gtgatgtcagctctcgacgaa

SEQ ID NO: 30 - DNA, 19 bp, artificial sequence
  misc_feature (1)..(19): reverse primer of SNP rs4354668
ctgccacctgtgctttgct

SEQ ID NO: 31 - DNA, 22 bp, artificial sequence
  misc_feature (1)..(22): forward primer of SNP rs4532
agcaagggagtcagaagacaga

SEQ ID NO: 32 - DNA, 23 bp, artificial sequence
  misc_feature (1)..(23): reverse primer of SNP rs4532
gtgttcagagtcctcatcttcct

SEQ ID NO: 33 - DNA, 19 bp, artificial sequence
  misc_feature (1)..(19): forward primer of SNP rs1800497
acggctccttgccctctag

SEQ ID NO: 34 - DNA, 20 bp, artificial sequence
  misc_feature (1)..(20): reverse primer of SNP rs1800497
ccttcctgagtgtcatcaac

SEQ ID NO: 35 - DNA, 19 bp, artificial sequence
  misc_feature (1)..(19): forward primer of SNP rs1800955
aggatcaactgtgcaacgg

SEQ ID NO: 36 - DNA, 20 bp, artificial sequence
  misc_feature (1)..(20): reverse primer of SNP rs1800955
tggtatctggcaaaacctcc

SEQ ID NO: 37 - DNA, 19 bp, artificial sequence
  misc_feature (1)..(19): forward primer of SNP rs4680
atccaagttcccctctctc

SEQ ID NO: 38 - DNA, 20 bp, artificial sequence
  misc_feature (1)..(20): reverse primer of SNP rs4680
ctttttccaggtctgacaac

SEQ ID NO: 39 - DNA, 18 bp, artificial sequence
  misc_feature (1)..(18): forward primer of SNP rs743572
gctccaggagaatctttc

SEQ ID NO: 40 - DNA, 20 bp, artificial sequence
  misc_feature (1)..(20): reverse primer of SNP rs743572
gacaatcactgtagtcttgg

SEQ ID NO: 41 - DNA, 20 bp, artificial sequence
  misc_feature (1)..(20): FAM labeled forward primer of 5HTT_VNTR
ggcgttgccgctctgaatgc

SEQ ID NO: 42 - DNA, 23 bp, artificial sequence
  misc_feature (1)..(23): reverse primer of 5HTT_VNTR
gagggactgagctggacaaccac

SEQ ID NO: 43 - DNA, 20 bp, artificial sequence
  misc_feature (1)..(20): FAM labeled forward primer of 5HTTstin2_VNTR
gctgtggacctgggcaatgt

SEQ ID NO: 44 - DNA, 24 bp, artificial sequence
  misc_feature (1)..(24): reverse primer of 5HTTstin2_VNTR
gactgagactgaaaagacataatc

SEQ ID NO: 45 - DNA, 18 bp, artificial sequence
  misc_feature (1)..(18): FAM labeled forward primer of DRD4_VNTR
ctgcagcgctgggaggtg

SEQ ID NO: 46 - DNA, 18 bp, artificial sequence
  misc_feature (1)..(18): reverse primer of DRD4_VNTR
aggaccctcatggccttg

SEQ ID NO: 47 - DNA, 30 bp, artificial sequence
  misc_feature (1)..(30): TET labeled forward primer of MAOA_VNTR
agcacgcgtgcctcagcctccttccccggc

SEQ ID NO: 48 - DNA, 30 bp, artificial sequence
  misc_feature (1)..(30): reverse primer of MAOA_VNTR
ccgagattcggcgggccctccgccttgcgc